(19) United States
(12) Patent Application Publication
(10) Pub. No.: US 2023/0248992 A1
(43) Pub. Date: Aug. 10, 2023

(54) Portable High Power Photobiomodulation Device with Measuring Ring

(71) Applicant: Leticia Muss Ross, San Diego, CA (US)

(72) Inventor: Leticia Muss Ross, San Diego, CA (US)

(21) Appl. No.: 17/668,053

(22) Filed: Feb. 9, 2022

(51) Int. Cl. A61N 5/06

(52) U.S. Cl. CPC A61N 5/0616; A61N 2005/0652; A61N 2005/0664; A61N 2005/0644

(57) ABSTRACT

A compact, high power, photobiomodulation (HPPBM) light device with distance measuring ring is disclosed. The light device is comprised of a hand-held cylinder with a plurality of high-powered light emitting diodes (LED), a calibrated measuring ring, a lithium-ion battery, and a universal serial bus (USB) charge port. The device combines HPPBM light with a measuring ring to ensure proper therapy. An object of the device is to maintain the reliability of the HPPBM therapy process by ensuring the light is positioned at a specific distance away from the skin at all times during self-treatment. Other embodiments can include various power levels and light spectrums for said LEDs.

### FIELD OF THE INVENTION

The invention relates to high-power, HPPBM therapy, formerly known as Phototherapy and Light Therapy. More specifically, it relates to a line of HPPBM light devices that combines HPPBM lights with a measuring ring or spacer to ensure consistent distancing guaranteeing a correct dose of the therapeutic light every session.

### BACKGROUND

HPPBM therapy can deliver therapeutic wavelengths of natural light to the body's skin and tissues not invasively. Such therapy, commonly referred to as light therapy, red light therapy, phototherapy, and HPPBM offers a wide range of health benefits that can include wound healing, pain relief and skin rejuvenation. Red-light therapy exposure can stimulate adenosine triphosphate within the body to boost collagen production and repair damaged tissues. One of the major benefits of red-light therapy is the effect on the energy center within the body's cells. This energy center is made up of mitochondria in the cells. Mitochondria produces the energy or fuel that our cells need to function and ultimately survive. As humans age, cells in the body become unwell, suffer from stress and can become slow to heal, inflamed or they simply die off. The body's cellular structure is the catalyst for most diseases and degenerative conditions. Red light therapy can increase nitric oxide, a compound that heals the cells and increases the blood flow which may help to stimulate healing and strengthen your immune system to defend against disease; regulate blood pressure and reduce inflammation. Nitric oxide is also crucial to the health of arteries. Red light therapy can help the mitochondria to produce the fuel or energy it requires for healthy cells and enhanced cell production. Red LED light can penetrate beyond the top layers of the skin to between eight and 10 millimeters which can influence the nervous system, metabolic processes as well as the immune system And because unhealthy cells are the cause of most diseases and degenerative conditions, it stands to reason that healthy cells can lead to better health, reduced stress levels and feelings of wellbeing. Infrared lights go deeper, penetrating up to 40 millimeters. It is usually applied to the patient's skin, scalp or directly to the blood intravenously. If the HPPBM light is applied to the skin it is typically provided to the patient's skin either with a wrap or lamp. Blue, blue-green and green lights HPPBM are commonly used to treat jaundiced babies. Because infant's skin is thin and the blood vessels are close to the surface, HPPBM infant devices are customized by using varying proportions of blue, blue-green and green lights. They generate very little heat and are safe to use relatively closer to the baby's skin. There is a need for a light device that combines HPPBM light with a measuring ring to maintain the consistency of dosing of the photo-medical light. The body's ability to combat injury, inflammation and skin conditions may also be enhanced. New areas of research into the benefits of HPPBM light therapy have been increasing. United States Patent No. US20160175609A1 granted to Catherine L. Dye and Markus P. Hehlen disclosed an LED panel for LED skin treatment. U.S. Pat. No. 6,488,698B1 granted to Henry H. Hyman disclosed a portable light treatment device inside a brief case. U.S. Pat. No. 7,131,990B2 granted to Vineet Bansal, Bryan Flaherty, and Chris Chung disclosed a phototherapy system that delivers light using mobile stands. None of these patents included a distance option to allow a user to maintain the proper distance when holding the light from the skin for optimal performance and consistent dosing.

### SUMMARY OF THE INVENTION

The device herein disclosed and described provides a solution to the shortcomings in the prior art through the disclosure of a compact and/or handheld, dermal, HPPBM light device with measuring ring (spacer) and/or using high power light. The present invention is a portable device that irradiates the skin penetrating layers of tissue up to 40 millimeters. An object of the device is to provide a hand-held HPPBM therapy device. The device comes with an array of high-powered HPPBM LEDs that project lights at one or more therapeutic wavelengths to promote healing within the body. High Power (LED diodes irradiating above 500 mW input) in a compact HPPBM device is novel, not found in prior small, compact or handheld devices, just present in very large and expensive machinery afforded just by clinics or hospitals. This is because high power lights (lasers and LEDs) generate a lot of heat, overheating the machines and deviating the wavelength that must have precision. The formal solutions for this heat problem, is the use of large and heavy mechanic cooling fans. This large cooling fans are unfit for compact devices. Unlike the existing compact handheld devices that have to use underpowered, low level light therapy (under 200 mW input) consequently needing to irradiate the lights for much longer (20 to 30 minutes per session), delivering wicker flux of light that does not penetrate the layers of tissue deep like to high-power lights and not reaching the joints, muscles or soft tissue being treated deeper than the skin surface. Lumina's proprietary technology solves the overheating issue without the need of bulky mechanical fans, guaranteeing the precision necessary for the wavelength while still using a compact housing. This breakthrough is all made possible by Lumina's technology built on industry-leading devices. This improved technology offers advances in chip architecture and light systems optimized for highest thermal and optical performance and stability. Other embodiments of the device can use other power levels and light spectrums.

Another object of the invention is to allow the device to be portable. The light device is small and has an onboard battery that can be recharged through a USB port. Other power sources can be used to drive the light through said USB port.

Another object of the device is to enhance HPPBM therapy by providing a means to help the user hold the device at a specific distance from the skin at all times. Having a consistent distance during treatment enhances the reliability to ensure a constant exposure at all times that can aid in measuring progress over time. This distance is maintained by a calibrated measuring ring at the head of the device. It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects, features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

FIG. 1 showing a perspective view of the device.

FIG. 2 showing a rear perspective view of the device.

FIG. 3 showing an exploded view of the device.

FIG. 4 showing a representative view of the device's method.

### DETAILED DESCRIPTION OF THE FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

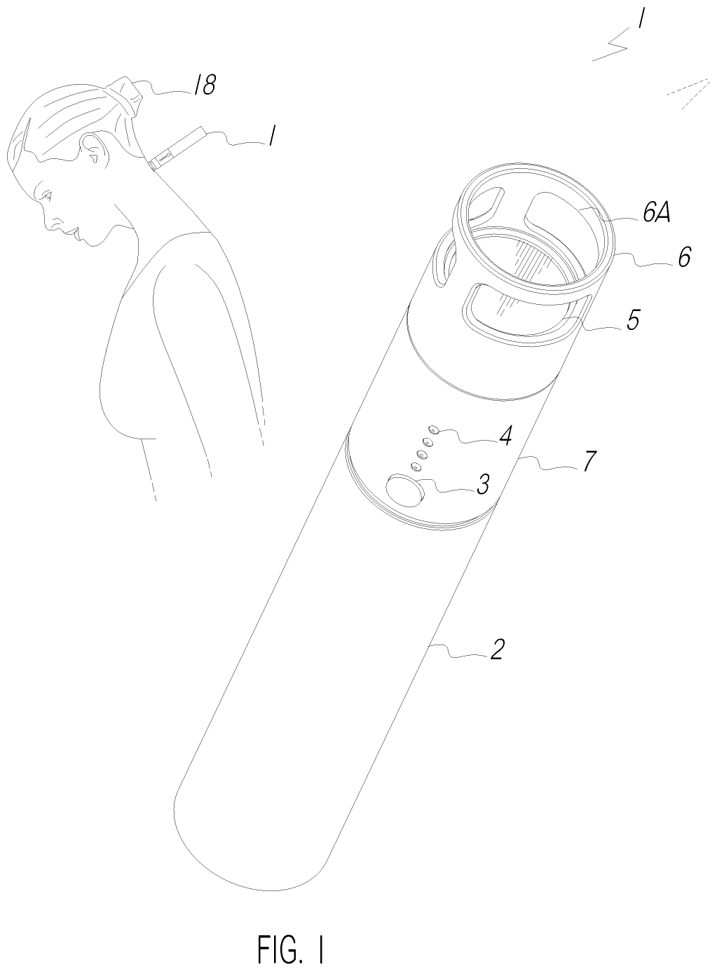

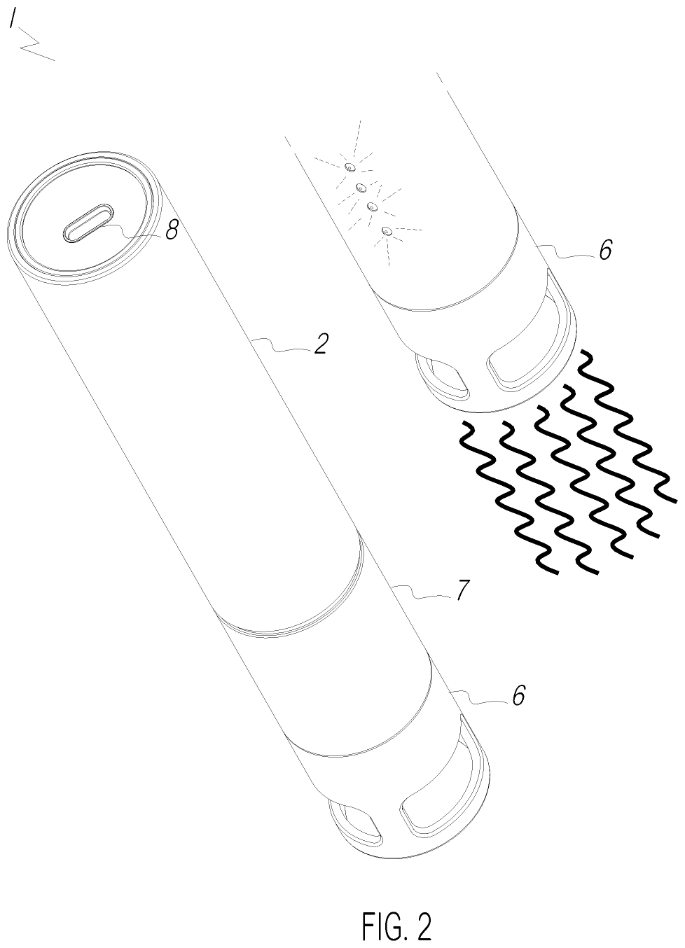

FIG. 1 shows a perspective view of a preferred embodiment of the HPPBM light device 1 and is comprised of a cylindrical-shaped, hand-held unit with casing 2 on one distal end and a distance measuring ring 6 on another distal end. The distance measuring ring 6 having rectangular apertures 6A configured radially and perpendicular to the device's longitudinal axis. The rectangular apertures 6A having a length that keeps said lens a healthy distance away from the distal end of the device ensuring proper use. Said distance measuring ring 6 is made of a rigid material that includes but is not limited to stainless steel, plastic and the like. Other embodiments may include distance measuring rings that are made of a rigid, yet flexible material such as but not limited to silicone. A lens 5 is disposed proximally within said distance measuring ring 6. The distance measuring ring 6 and the casing 2 being connected by means of central ring 7 which also has a plurality of status LEDs 4 and a power switch 3. Said status LEDs 4 displaying colors that allow the user to know when the device is on and when the device is being charged. In still other embodiments, the status LEDs 4 can serve to notify the user that the proper distance from a distal end of the device is being maintained. The HPPBM light device 1 is made of a rigid material that includes but is not limited to aluminum, stainless steel, other metals, silicone, plastic and the like while lens 5 is made of transparent glass or plastic material. The figure also showing user 18 receiving treatment from the HPPBM light device 1 with distal measuring piece 6 being pressed onto user 18's skin thereby allowing the HPPBM light to be at a healthy distance for maximum treatment. FIG. 2 shows case 2 having USB power port 8.

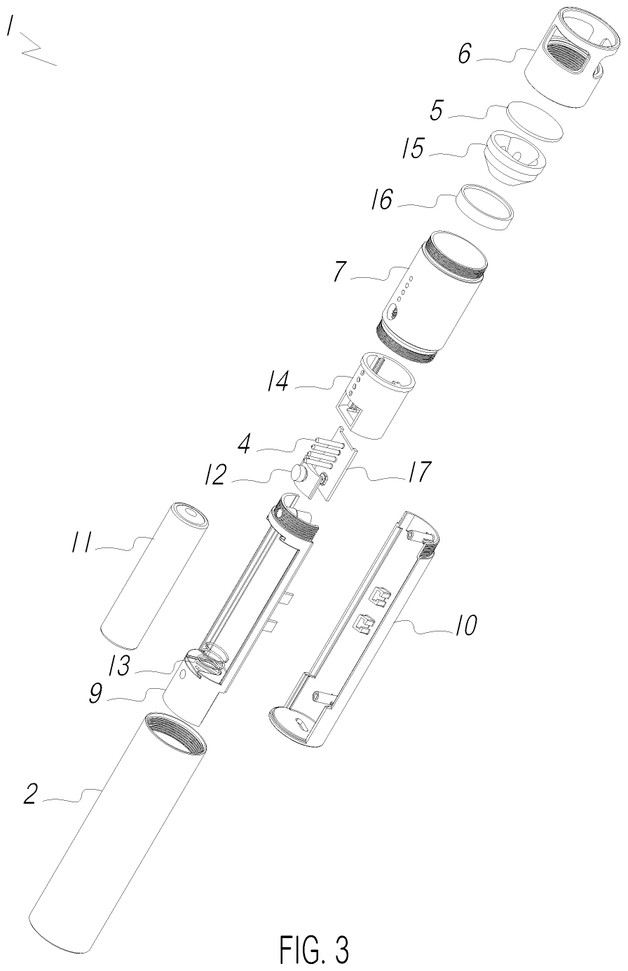

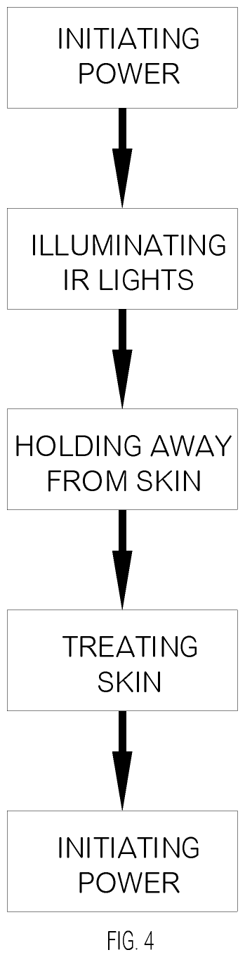

FIG. 3 shows an exploded view of the HPPBM light device 1 with central ring 7 being hollow with HPPBM light cluster 15, holding ring 16 and lens 5 and hollow control ring 14 there inside. Said control ring 14 configured to receive control board 17 with a plurality of status LEDs 4 and power switch. The aforementioned HPPBM light cluster 15 having at least but not limited to three high-powered HPPBM lights having greater than 1 watt input and are in the red and infrared spectrum, two of which are at least ˜660 nm and one is at least ˜850 nm. Other embodiments of the device can use other power levels and light spectrums including various wattage levels and colors etc. Casing 2 also being hollow and having half cradles 9 and 10 along with terminal spring 13 to receive rechargeable battery 11 being comprised of those such as lithium ion, nickel hydride etc. FIG. 4 showing a representative view of the device's method that include but are not limited to: initiating the power switch; illuminating HPPBM light cluster; placing device against a user's body with the distance measuring ring holding it away from the skin; and HPPBM light cluster treating the skin.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A photobiomodulation light device comprised of the following parts:
a) a casing;
b) photobiomodulation light cluster;
c) a distance measuring ring; and
d) a central ring.

2. The photobiomodulation light device of claim 1, wherein the casing having half cradles with terminal spring and rechargeable battery.

3. The photobiomodulation light device of claim 1, wherein the photobiomodulation light cluster is high-powered.

4. The photobiomodulation light device of claim 1, wherein the distance measuring ring having rectangular apertures and a lens.

5. The photobiomodulation light device of claim 1, wherein the central ring having status LEDs, a power switch, a photobiomodulation light cluster, holding ring and control board.

6. A method of treating skin with photobiomodulation light. The method comprising the following steps:
a) providing the photobiomodulation light device of claim 1;
b) initiating the power switch;
c) illuminating photobiomodulation light cluster;
d) placing device against a user's body with the distance measuring ring holding it away from the skin; and
e) treating the skin with the photobiomodulation light cluster.
